(19) United States
(12) Patent Application Publication
(10) Pub. No.: US 2003/0113931 A1
(43) Pub. Date: Jun. 19, 2003

(54) Ammonia and ammonium sensors

(76) Inventors: Li Pan, Tampa, FL (US); Ramesh Wariar, Tampa, FL (US); Vance Maples, St. Petersburg, FL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 10/024,170

(22) Filed: Dec. 14, 2001

(51) Int. Cl. G01N 33/00

(52) U.S. Cl. 436/113; 436/111; 436/164; 436/165; 436/167; 422/55; 422/58; 422/82.05

(57) ABSTRACT

A sensor which senses concentrations of a component of a fluid. In a dialysis system, the sensor can monitor the total ammonia and ammonium concentration in dialysate. The sensor has an optical indicator, such as an ammonia sensitive membrane, positioned in direct contact with the dialysate fluid when in use. The ammonia sensitive membrane has a variable optical indication, such as a variable color change, in relation to the concentration of ammonia in the dialysate. An optical reader reads the color change of the membrane to measure the total ammonia and ammonium concentration in the dialysate. The sensor can be used in the dialysis system to monitor the effectiveness of sorbants which remove ammonia/ammonium which is produced from an enzyme reaction with urea, the urea being removed from a patient during dialysis treatment.

### BACKGROUND OF THE INVENTION

The present invention generally relates to sensors which sense a component of a fluid, and methods of sensing fluid components. More specifically, the present invention relates to sensors which sense ammonia and ammonium in solutions.

In a number of contexts it is desirable to sense ammonia, ammonium, or total ammonia and ammonium in solutions. For example, in certain medical treatments, the level of ammonia or ammonium of a solution can be a critical issue. An example of such a medical treatment is dialysis, such as peritoneal dialysis and hemodialysis.

Peritoneal dialysis utilizes a dialysis solution or dialysate, which is infused into a patient's peritoneal cavity. The dialysate contacts the patient's peritoneal membrane in the peritoneal cavity. Waste, toxins, and excess water pass from the patient's bloodstream through the peritoneal membrane and into the dialysate. The transfer of waste, toxins, and water from the bloodstream into the dialysate occurs by diffusion and osmosis because there is an osmotic gradient across the peritoneal membrane. The spent dialysate is drained from the patient's peritoneal cavity to remove the waste, toxins, and water from the patient. Fresh dialysate is then provided to the peritoneal cavity.

One waste component removed from the patient by the dialysate is urea. The spent dialysate containing urea can be discarded, for example disposed down a drain. Fresh dialysate is then supplied to the patient to continue the dialysis therapy. However, a rather large amount of dialysate, such as 30 liters or more, is commonly used during the dialysis therapy because the fluid is disposed to the drain.

Alternatively to disposing such a large amount of fluid, the spent dialysate can be regenerated into fresh dialysate. The spent dialysate can be regenerated, in part, by removing the urea and other undesired components from the dialysate. Urea can be removed from the dialysate by chemically converting the urea to mainly ammonia, ammonium, and bicarbonate. The ammonia and ammonium are then removed from the dialysate.

Of course, if the ammonia and ammonium are not removed from the dialysate it will remain in the dialysate. This is an undesirable condition. Therefore, there is a need to monitor the concentrations of ammonia and ammonium in the dialysate after the urea conversion and ammonia/ammonium removal processes. Monitoring the ammonia/ammonium concentration in the dialysate can provide an indication of the effectiveness of ammonia/ammonium removal.

Another type of dialysis treatment is hemodialysis. Hemodialysis utilizes the patient's blood to remove waste, toxins, and excess water from the patient. In a hemodialysis procedure, the patient is connected to a hemodialysis machine and the patient's blood is pumped through the machine. As blood passes through a dialyzer in the hemodialysis machine, waste (urea), toxins, and excess water are removed from the patient's blood and the blood is infused back into the patient. The urea and other components removed from the blood pass through the dialyzer membrane into the dialysate on the other side of the dialyzer.

In one type of hemodialysis, the spent dialysate can be regenerated. An existing regenerative hemodialysis system uses a Redy cartridge by Sorb Technology, Inc., Oklahoma City, Okla. The Redy cartridge converts urea in the spent dialysate to ammonia, ammonium, and bicarbonate and then zirconium phosphate removes the ammonium. The fresh dialysate is returned to the dialyzer for further adsorption from the blood. Adsorption of the ammonium is limited by the quantity of the zirconium phosphate.

As noted above, it is desirable to monitor the ammonia and ammonium levels of the fluid. A system using the Redy cartridge uses reagent paper strips to measure the fluid ammonia/ammonium content. The reagent paper is manually dipped into an open reservoir of the fluid. The reagent strip reacts with the ammonia/ammonium in the fluid and over time changes color due to the ammonia/ammonium. The color change of the reagent strip is manually viewed by the person handling the reagent strip. The color of the strip is visually compared to a color chart to determine the concentration of ammonia/ammonium.

There are several drawbacks with using reagent strips to measure ammonia and ammonium. The reagent strips require the operator to manually dip the paper strip into the reservoir. Because the strip is manually dipped into the fluid, the fluid is obviously open to the surrounding environment. The open fluid can be subject to contamination from the environment. Furthermore, the color change of the strip is viewed by the naked eye. The subtle color changes at different ppm ammonia/ammonium concentrations are subject to the viewer's interpretation and description. Therefore, the measurement may not be accurate. Furthermore, there is a reaction time after dipping the strip into the fluid before the strip changes color. During this time delay ammonia/ammonium continues to accumulate in the fluid. Thus, the actual ammonia/ammonium fluid concentration is actually higher than the concentration shown by the color of the strip. Also, the reagent strip is a single use indicator because the color change is not reversible. Overall, the reagent strip measurement is a twenty year old technology which requires the patient or operator to dip, wait, and evaluate by visual perception weak color changes in a system where fluid contamination from the environment could be a problem during therapy.

Another attempt to measure ammonium content of fluid in a fluid path uses a diffusion technique. In the diffusion technique, the fluid path has an opening which is covered by a Teflon gas permeable membrane. The fluid in the fluid path contacts the gas permeable membrane at the fluid opening. Ammonia gas inside of the fluid passes through the fluid opening and diffuses through the gas permeable membrane to outside of the fluid path. After passing through the gas permeable membrane, the ammonia outside of the fluid path is directed to a pre-wetted ammonium sensitive chemical strip. The pre-wetted chemical strip converts the ammonia to ammonium. The chemical strip then changes color as it dries according to the ammonium concentration. The color change of the chemical strip is used to determine the ammonium concentration.

However, the diffusion technique has drawbacks. For example, only a very small portion of ammonia/ammonium in the fluid passes through the diffusion filter to the chemical strip. Typically, the amount of ammonia in the fluid at a physiological pH level is as little as 1% of the total ammonia/ammonium. Thus, the color change of the chemical strip and the measurement is based on a very small amount of diffused ammonia. This can lead to inaccurate measurements. Additionally, the Teflon diffusion filter needs to be rather strong to prevent fluid leakage through the opening while permitting ammonia diffusion. Also, contaminates may enter the fluid path from the environment through the diffusion filter into the fluid path. Furthermore, the chemical strip changes color in one direction only, i.e. the color change is not reversible. Once the color of the chemical strip is set by exposure to an ammonia concentration, the strip cannot be used to measure a lower ammonia concentration because the strip color will not change. Thus, the chemical strip is a single use measurement device and is not reusable. Further, the chemical strip must be wetted to react and dried to read the color change.

Another attempt to monitor ammonia and ammonium in a fluid path uses an electrical conductivity technique. This technique uses a fluid flow injection analyzer device and an ultra-pure water source. The electrical conductivity of the ultra-pure water changes in relation to the amount of ammonia and ammonium in solution. The conductivity of the ultra-pure water is measured before and after the injection of the test fluid and the conductivity measurements are used to calculate the total ammonia and ammonium quantity in the fluid. However, this is an expensive measurement technique and requires calibration and calibration solutions for the conductivity measurements. Such a system is not suitable for monitoring ammonia/ammonium in dialysis systems.

Thus, needs exist for commercially viable ways and devices to measure ammonia and ammonium in fluids. Such needs particularly exist for medical devices and procedures.

### SUMMARY OF THE INVENTION

Generally, the present invention provides new sensors, methods of sensing, and sensing systems. The invention particularly pertains to optical sensing of total ammonia and ammonium in a fluid used for peritoneal dialysis. However, the principles of the invention can be used for sensing fluid components other than ammonia and ammonium. The present invention can also be practiced outside of peritoneal dialysis, for example, dialysis in general, hemodialysis, and other medical and non-medical applications. In a preferred embodiment, the invention provides a total ammonia and ammonium sensor in a peritoneal dialysis system.

In an embodiment of the present invention, a sensor for sensing ammonia in a fluid is provided. The sensor includes a fluid flow path having an optical window; a membrane positioned within the fluid flow path, the membrane having a color indicative of the concentration of the ammonia in the fluid; and an optical reader positioned outside of the fluid flow path so as to identify the color of the membrane through the optical window.

In an embodiment, the membrane is a hydrophobic membrane.

In an embodiment, the sensor further includes a fluid pH conditioner in the fluid flow path.

In an embodiment, the sensor further includes a fluid parameter sensor having an output signal provided to a processor. The processor utilizes the output signal of the fluid parameter sensor to determine the ammonia concentration.

In an embodiment, the parameter sensor senses a parameter selected from the group consisting of a temperature, pH, and combinations thereof.

In an embodiment, the fluid flow path is a portion of a dialysis system flow path.

In an embodiment, the optical window comprises a flexible sheeting.

In an embodiment, the sensor further includes an infrared emitter connected to a processor; a first color emitter connected to the processor; a second color emitter connected to the processor; and a photo-detector connected to the processor.

In an embodiment, a processor determines a total ammonia and ammonium concentration of the fluid.

In another embodiment of the present invention, a sensor for a dialysis system is provided. The sensor includes a fluid container; a membrane positioned inside of the fluid container and having a variable optical property; and an optical reader positioned outside of the fluid container in a sensing relationship with the membrane.

In an embodiment, the fluid container is a disposable unit for use in a single dialysis therapy treatment.

In an embodiment, the membrane is a hydrophobic membrane.

In an embodiment, the membrane is a calorimetric ammonia sensitive membrane.

In an embodiment, the optical reader is a colorimetric reader.

In an embodiment, the sensor further includes a fluid pH adjustor upstream of the membrane.

In an embodiment, the sensor further includes a fluid temperature sensor at the fluid container.

In an embodiment, the sensor further includes a processor connected to the optical reader. The processor has an output indicative of a fluid parameter sensed by the sensor.

In an embodiment, the output of the processor is indicative of one of ammonia in the fluid flow path, ammonium in the fluid flow path, total ammonia and ammonium in the fluid flow path, and combinations thereof.

In another embodiment of the present invention, a sensor for sensing concentrations of a component of a fluid of a dialysis system in which at least a portion of a fluid flow path of the dialysis system is closed to surrounding environment is provided. The sensor has an optical indicator positioned within the closed fluid flow path and in direct contact with the fluid when the optical indicator is in use. The optical indicator has a variable optical characteristic of the concentration of the component when the optical indicator is in direct contact with the fluid. The sensor also has an optical reader located outside of the closed fluid flow path and so positioned and arranged to detect the optical characteristic of the optical indicator. The optical reader generates an output signal indicative of the optical characteristic of the optical indicator.

The variable optical indication may have variable colors. The optical sensor may have an ammonia sensing membrane. The optical reader may be a colorimetric sensor. The sensor may also include a processor which receives the output signal of the optical reader and determines an ammonia concentration based at least in part on the output signal of the optical reader.

In another embodiment of the present invention, an ammonia sensor for a dialysis system is provided. The ammonia sensor includes a disposable unit having a fluid flow path; a ammonia sensitive membrane inside of the fluid flow path in the disposable unit; and a membrane reader positioned outside of the fluid flow path in sensing relationship with the membrane.

In an embodiment, the membrane is a colorimetric ammonia sensitive hydrophobic membrane.

In an embodiment, the membrane reader is a colorimetric reader.

Another embodiment of the invention provides a method of sensing ammonia in a dialysis system. The method includes providing an ammonia sensitive device inside of a fluid flow path having a fluid inlet and a fluid outlet; flowing dialysate through the fluid flow path; allowing the ammonia sensitive device to contact dialysate located in the fluid flow path; causing a color of a portion of the ammonia sensitive device to change in response to a concentration of ammonia in the dialysate; and identifying the color of the ammonia sensitive device from outside of the fluid flow path.

The method may further include the determining a total ammonia and ammonium concentration of the dialysate based at leased in part on the color of the ammonia sensitive device. The step of providing an ammonia sensitive device may further include the step of providing a hydrophobic ammonia sensitive membrane inside of the fluid flow path. The method may also include the step of adjusting a pH of the dialysate upstream of the ammonia sensitive device or measuring the fluid pH. The determining step may further include the step of determining the total ammonia and ammonium concentration of the dialysate based at least in part on the adjusted or measured pH. The method may further include measuring a temperature of the dialysate, and determining the total ammonia and ammonium concentration of the dialysate based at least in part on the measured temperature.

In another embodiment of the present invention, a method of performing dialysis is provided. The method includes the steps of removing waste from a patient using dialysate fluid; positioning in the dialysate fluid a membrane that changes a parameter in relation to the level of a component in the dialysate fluid; and identifying the change in the parameter of the membrane.

In an embodiment, the contacting step further includes contacting an ammonia sensitive membrane with the dialysate fluid.

In an embodiment, the step of changing a parameter of the membrane further includes changing an optical parameter of the membrane.

In an embodiment, the sensing step further includes identifying a color of the membrane.

In an embodiment, the method further includes the step of treating the dialysate fluid prior to the step contacting the membrane with the dialysate fluid.

In an embodiment, the treating step further includes adjusting a pH of the dialysate fluid.

In an embodiment, the step of removing waste further includes performing peritoneal dialysis.

In an embodiment, the step of removing waste further includes performing hemodialysis.

In an embodiment, the component is ammonia.

In an embodiment, the component is ammonium.

In yet another embodiment of the present invention, a method of performing dialysis is provided. The method includes removing waste from a patient using dialysate fluid and thereby forming spent dialysate; positioning in the spent dialysate an ammonia sensitive member which has a characteristic that changes in relation to the level of ammonia in the spent dialysate; and identifying a change in the characteristic of the ammonia sensitive member.

In an embodiment, the ammonia sensitive member is a membrane.

In an embodiment, the characteristic of the member is color.

In an embodiment, the removing waste step further includes performing peritoneal dialysis.

In an embodiment, the removing waste step further includes performing hemodialysis.

An advantage of the present invention is to provide improved sensors, particularly, ammonia and ammonium sensors.

Another advantage of the present invention is to provide improved methods of sensing, particularly ammonia and ammonium.

Yet another advantage of the present invention is to provide improved sensing systems, particularly ammonia and ammonium.

A further advantage of the present invention is to provide continuous monitoring of a substance during dialysis treatment.

Yet another advantage of the present invention is to provide a rapid response, low cost, effective, and accurate sensor.

An even further advantage of the invention is to monitor the effectiveness of a sorbant cartridge used in a regeneration dialysis system.

Yet still another advantage of the invention is that the sensor has a reversible color change capability and is reusable.

Furthermore, an advantage of the invention is that the sensing membrane is gamma sterilizable.

Further still, another advantage of the invention is that the sensor can be constructed in two parts, including a disposable color changing membrane and a color reader instrument.

Additional features and advantages of the present invention are described in, and will be apparent from, the following Detailed Description of the Invention and the figures.

### BRIEF DESCRIPTION OF THE FIGURES

FIG. 1 is a schematic diagram of a sensor according to the principles of the present invention.

FIG. 2 is another schematic diagram of the sensor of FIG. 1.

FIG. 3 is a schematic diagram of a control circuit for the sensor of FIG. 1.

FIG. 4 is a timing diagram of the sensor of FIG. 1.

FIG. 5 is a partial schematic diagram of a peritoneal dialysis system having a sensor according to the present invention.

FIG. 6 shows a pH adjuster of a sensor according to the invention.

FIG. 7 shows a disposable cassette having the pH adjuster of FIG. 6.

FIG. 8 shows another disposable cassette having another pH adjuster within the cassette.

### DETAILED DESCRIPTION OF THE INVENTION

The present invention generally relates to sensors which sense a component of a fluid, and methods of sensing fluid components. More specifically, the present invention relates to sensors and methods of sensing ammonia and ammonium in solutions, preferably medical solutions. In an embodiment, the present invention relates to sensors for use with dialysis systems. Although an embodiment of present invention will be described in the context of a total ammonia and ammonium sensor in a peritoneal dialysis system, the present invention is not limited only to such an embodiment or to peritoneal dialysis treatment.

In an embodiment, the present invention provides a total ammonia and ammonium sensor (TAAS) for aqueous solutions. The sensor can have a colorimetric hydrophobic ammonia sensing membrane and a colorimetric reader. The ammonia sensing membrane is placed in direct contact with the solution. Ammonia gas is highly soluble in the solution and can be quantified by contacting the hydrophobic ammonia sensing membrane which changes color based on the quantity of ammonia gas diffused into the membrane. Accordingly, ammonia in the solution penetrates the membrane and the membrane changes color in relationship to the amount of ammonia. The calorimetric reader reads the color of the membrane. A determination of the total ammonia and ammonium (TAA) in the solution is made based on the color reading.

The sensor may also have a pH indicator or a pH conditioner, a temperature sensor, and a mathematical model that calculates the ammonia and ammonium content utilizing the parameters of ammonia concentration, pH, and temperature.

Numerous measurements at various ammonia concentrations can be made using the membrane because the color changes of the membrane are reversible, i.e. the membrane changes color with both increases and decreases of ammonia concentrations. A tri-wavelength optical sensor located outside of the fluid flow path measures the color of the membrane through a transparent window. Based on the colorimetric readings, pH of the fluid, and temperature of the fluid, the total ammonia and ammonium can be determined.

The total ammonia and ammonium sensor is based on non-contact measurement by the optical colorimetric reader. A sterile sensing membrane can be placed inside a fluid flow path and the optical colorimetric reader can be embodied in an instrument which reads the color change through a window in the flow path conduit. Accordingly, it is practical to make the sensing membrane part of a sterile disposable unit having the fluid flow path in which the disposable unit interfaces with the optical reader of the non-disposable instrument.

Referring now to the drawings, one sensor 10 according to the invention is shown schematically in FIG. 1. The sensor 10 has an optical indicator 12 and an optical reader 14. The optical indicator 12 is positioned within a fluid flow path 16 during use such that the optical indicator 12 is in direct contact with the fluid in the flow path 16. The fluid flow path 16 is shown by way of example as being in a housing 18 and having an inlet 20 and an outlet 22. The left side of the fluid flow path 16, as viewed in FIG. 1, is covered with a membrane or sheeting 24, for example, which is sealed to the housing 18. The sheeting 24 is an optical window because it is at least substantially transparent to optical signals relative to the optical reader 14 and the optical indicator 12.

The optical indicator 12 is sensitive to a component of the fluid in the fluid path 16 to be sensed. A property of the optical indicator 12 is that the indicator 12 reacts to the fluid component and changes an optical parameter depending on the concentration of the component in the fluid. In other words, the optical indicator 12 has an optical indication that varies with respect to the amount of the component in the fluid that contacts the indicator 12. Examples of the optical indication include color, reflectivity, fluorescence, adsorption, and any other optical indication.

In a preferred embodiment, the optical indicator 12 is a sensing membrane which changes color in relationship to changes in the concentration of the component to be measured in the fluid. As the concentration of the component in the fluid increases, the color of the membrane 12 changes in a first direction along a color spectrum, and as the concentration of the component in the fluid decreases, the color of the membrane 12 changes along the color spectrum in a reverse direction relative the first direction. Preferably, the color change of the membrane 12 is continuous and automatically reversible (the color can move in either direction along the color spectrum) in response to the component concentration.

The optical reader 14 detects or reads the optical indication of the optical indicator 12. The optical reader has an output signal indicative of the optical indication of the optical indicator 12. The output signal of the optical reader 14 varies in relationship to any change of the optical indication due to a change in the concentration of the component to be sensed in the fluid. In this manner, the optical sensor 10 can not only detect the presence of the component in the fluid but also measure the concentration of the fluid component because of the correlation between the component concentration, the optical indication of the optical indicator 12, and the optical reader 14.

The optical reader 14 is positioned in a reading relationship with respect to the optical indicator 12, and is preferably positioned outside of the fluid path 16. The optical reader 14 does not contact the fluid in the fluid path 16 and is thus, a non-invasive measuring device. A detection signal emanates from the optical reader 14 and is directed toward the optical indicator 12. The detection signal hits the optical indicator 12, reflects off of the optical indicator 12, and is read by the optical reader 14. In this manner, the optical reader 14 reads the optical indication of the optical indicator 12.

FIG. 1 shows one embodiment of the optical reader 14, in which the detection signal is reflected off of the optical indicator 12; however, other embodiments are within the scope of the invention. For example, the detection signal can be read after it passes through the optical indicator 12 rather than being reflected by the optical indicator 12. In such an embodiment, a signal generator and a signal detector of the optical reader 14 would be positioned on opposite sides of the optical indicator 12.

The structure of the sensor 10, particularly the optical indicator 12 being in direct contact with the fluid, and the optical reader 14 not being in contact with the fluid, provides advantages. For example, the direct contact of the optical indicator 12 with the fluid provides an efficient and accurate sensing of the fluid component. The sensor 10 can continuously monitor the component concentration with rapid response. Furthermore, the non-invasive optical reader 14 prevents contamination of the fluid by the optical reader 14.

The optical indicator 12 of the total ammonia and ammonium sensor 10 is a hydrophobic membrane which is sensitive to ammonia. A preferred membrane is disclosed in U.S. patent application Ser. No. ______ titled “Hydrophobic Ammonia Sensing Membrane,” which is being filed herewith, the entire disclosure of which is incorporated herein by reference.

As described in that patent application, the membranes are capable of sensing a gas dissolved in solution, such as ammonia dissolved in dialysate solution. The ammonia sensing membranes includes a hydrophobic membrane that has a microporous structure and a pH sensitive dye embedded within the microporous structure of the membrane. In this regard, the ammonia sensing membrane is capable of selectively detecting gaseous phase ammonia as the pH sensitive dye which is embedded within a surface of the microporous membrane structure composed of strands is colorimetrically active in the presence of gaseous phase ammonia. In other words, the dye changes color in response to the ammonia.

As further described in the application, the membranes can include a variety of different and suitable material components and can be produced in a variety of suitable manners. In an embodiment, the membranes include a membrane material that is hydrophobic in nature (e.g., a hydrophobic membrane material). The hydrophobic membrane material can be composed of a variety of different and suitable materials. In an embodiment, the membrane material includes polypropylene, polytetrafluoroethylene (“PTFE”), polyvinylidene difluoride (“PVDF”), fluorinated ethylene propylene polymers (“FEP”), acrylic-based polymeric compounds, acrylic-based fluorinate polymers, copolymers thereof, combinations thereof and other suitable polymeric compounds.

As also described in the application, the membranes preferably include a pH sensitive dye. The pH sensitive dye of the present invention can include a variety of different and suitable materials including, for example, bromophenol blue, bromothymol blue, methyl yellow, methyl orange, 2,4-dinitrophenol, 2,6-dinitrophenol, phenol red, mixtures thereof and other suitable dye sensitive materials.

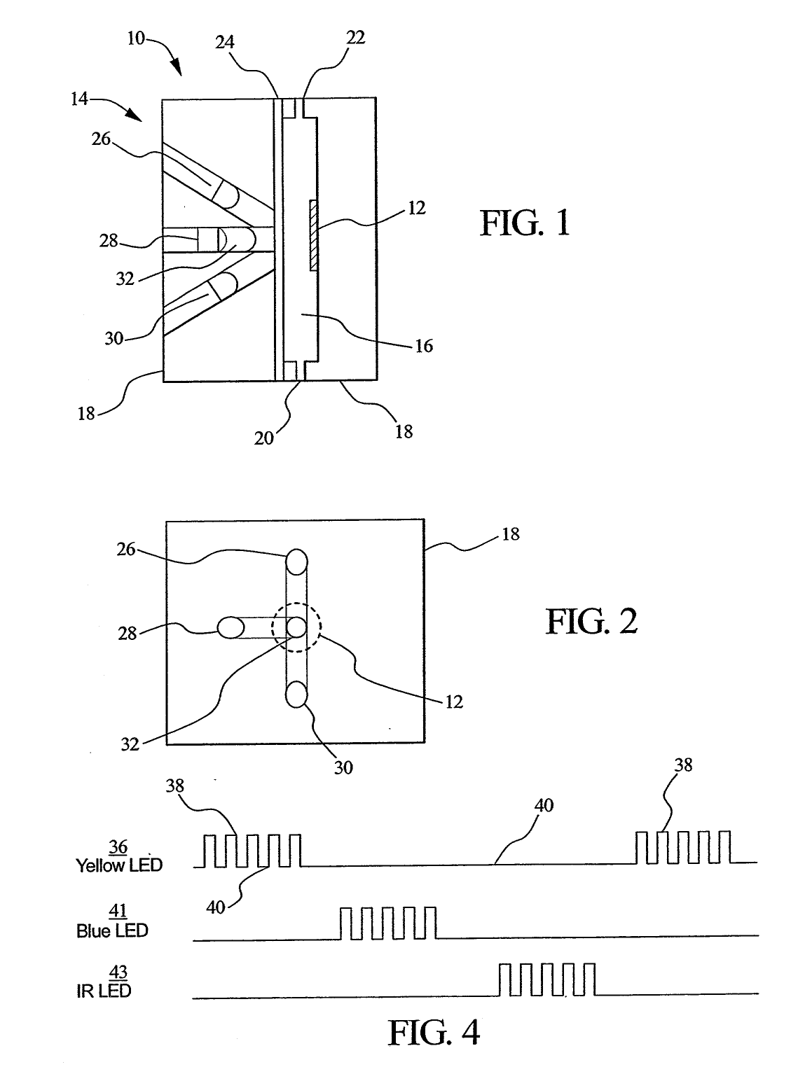

Referring back to FIGS. 1 and 2, water-soluble ammonia gas (NH₃) diffuses through the hydrophobic membrane 12 and changes the color of the membrane 12. The ammonia sensing membrane 12 changes color in relationship to changes in the ammonia concentration of the fluid. As the ammonia concentration of the fluid increases, the color of the membrane 12 changes in a first direction along a color spectrum, and as the ammonia concentration of the fluid decreases, the color of the membrane 12 changes along the color spectrum in a reverse direction relative the first direction. The color change of the membrane 12 is continuous, automatic, and reversible (the color can move in either direction along the color spectrum) in response to the ammonia concentration.

Examples of the membrane color for one membrane 12 according to the invention include yellow at 0 ppm ammonia, light blue at 10 ppm ammonia, and deep blue at 400 ppm ammonia. The membrane color change is highly sensitive to changes in ammonia concentration. By way of example, one ammonia sensing membrane 12 has a sensitivity of about 0.1 ppm. Also, the membrane 12 has been tested for response time to change color and has been shown to respond (change color) to ammonia concentration change within 20 seconds to one minute, for example.

Referring to FIGS. 1 and 2, in an embodiment, the optical reader 14 is a tri-wavelength optical transducer. The optical transducer, by way of example in this embodiment, has a yellow LED (light emitting diode) 26, an infrared LED 28, and a blue LED 30. Other embodiments may utilize more or fewer LED's, as desired. The yellow, infrared, and blue LED's 26, 28, 30 are preferably positioned at an angle of about 45° relative to the hydrophobic membrane 12, although other positions or angles can be used. Preferably, all of the LED's 26, 28, 30 are focused on the same optical field (portion) of the surface of the membrane 12. This provides for consistent sensor readings. One or more photo-detectors 32 of the optical reader 14 receives the light signals emitted by the LED's 26, 28, 30 and scattered off of the membrane 12. Other embodiments can use any suitable detector. One version of the sensor 10 has been designed to measure the optical absorbance changes of the membrane 12 up to solution NH3 concentrations at 100 ppm. One embodiment of the sensor 10 has an operating range for measuring ammonia concentrations at about 1-100 ppm. Another embodiment of the sensor 10 has an operating range for measuring ammonia concentrations at about 1-20 ppm.

Referring now to FIG. 3, the schematic diagram shows an example of a control circuit 34 for the sensor 10. In this embodiment, the sensor 10 is computer controlled by the control circuit 34 which provides driving signals to the LED's 26, 28, 30 to send light signals toward the hydrophobic membrane 12. The photo-detector 32 provides one or more signals indicative of the membrane color. The control circuit 34 then processes the signal(s) from the photo-detector 32 and produces an output indicative of the component of the fluid that is sensed. The sensor control circuit 34 can be constructed and programmed to determine and output any desired information based on the sensed fluid parameter. For example, the control circuit 34 can determine the total ammonia and ammonium concentration in the dialysate, and the individual concentrations of ammonia and ammonium. The output of the control circuit 34 can be in any desired form. Furthermore, the output can be numeric, graphic, or an audible alarm, for example.

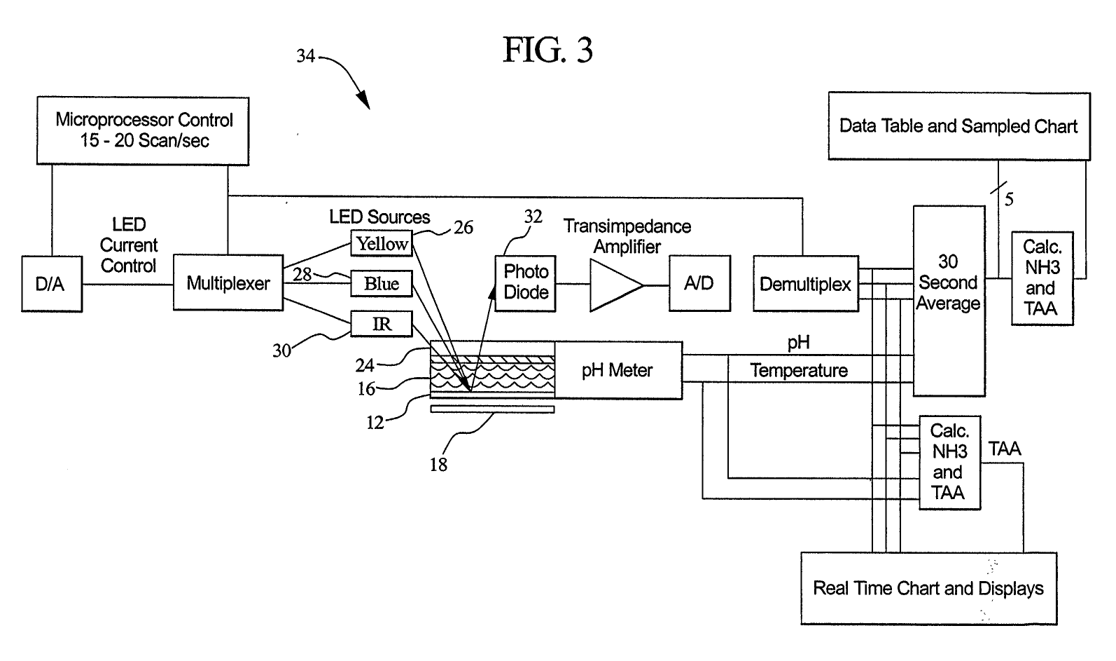

Referring to FIG. 4, an exemplary timing diagram shows the preferred driving signals 36, 41, 43 supplied to the LED's 26, 28, 30 by the control circuit 34. The FIG. 4 timing diagram shows the driving signals 36, 41, 43 in a multiplexing and de-multiplexing mode. Referring to the yellow LED driving signal 36, the yellow LED 26 is repeatedly turned ON (see reference numeral 38) and OFF (see reference numeral 40 ) through a defined yellow LED actuation time period. The controller similarly drives the blue LED 30 with a driving signal 41 through a blue LED actuation time period, and the infrared LED 28 with a driving signal 43 through an infrared actuation time period. A time period of all LED's 26, 28, 30 being OFF occurs between each yellow, blue, and infrared actuation time periods. The output signal of the photo-detector 32 is a voltage responsive to the ON/OFF of the yellow, blue, and infrared LED's 26, 28, 30 which is processed by the control circuit 34. The ON/OFF cycle of the LED's 26, 28, 30 is continuously repeated by the control circuit 34 during operation of the sensor 10.

In this embodiment, the infrared LED 28 is used to provide a baseline measurement for comparison to the signals from the yellow and blue LED's 26, 30. The reading taken from the infrared signal provides a transmissibility reading because infrared light from the infrared LED 28 is not affected by any color change of the membrane 12. Further in this embodiment, the yellow and blue LED's 26, 30 are used to detect the color and any color change of the membrane 12 because the yellow LED signal and the blue LED signal are affected by the membrane color. The time period of all LED's 26, 28, 30 being OFF can be used to determine if the photo-detector 32 is operating correctly. During the all LED OFF time periods, the output of the photo-detector 32 should be at a predetermined known voltage, such as 0 volts. Preferably, the control circuit 34 operates the yellow, blue, and infrared LED's 26, 28, 30 at a frequency which minimizes or avoids any effects of ambient light reaching the photo-detector 32, for example, about 2,000 htz.

Referring back to FIG. 3, the control circuit 34 may also be connected to other sensors or devices. Such sensors or devices may provide inputs to the control circuit 34 for the processing, for example, processing of the photo-detector signal to determine the total ammonia and ammonium concentration. Total ammonia and ammonium (TAA) can be determined primarily by three parameters: 1) NH₃ or NH₄⁺, 2) solution pH, and 3) solution temperature. Accordingly, in an embodiment of the invention, a pH sensor and/or a temperature sensor may be provided to sense the pH and/or temperature of the dialysis fluid. Ammonia and ammonium equilibrium correlations can be used to determine the TAA. The outputs of the fluid pH and temperature sensors are input into the controller and utilized in the processing to determine the total ammonia and ammonium in the dialysate, in this embodiment, The percent concentration of ammonia can be determined by the following equations.

ammonia %={1/[1+10exp(pKa−pH)]}×100 Equation 1.

pKa=0.09+[2729.9/(t+273)] Equation 2.

In Equation 2, t is the temperature of the fluid, ° C.

An embodiment of the sensor invention in a peritoneal dialysis system will now be more thoroughly described. In this embodiment, the sensor 10 shown in FIGS. 1 and 2 is a total ammonia and ammonium sensor. The sensor 10 is used to detect and measure the total ammonia and ammonium concentration in a dialysis solution during renal therapies. The sensor 10 can automatically and continuously monitor the total ammonia and ammonium. Of course, the sensor 10 can also be operated to periodically monitor the total ammonia and ammonium as desired.

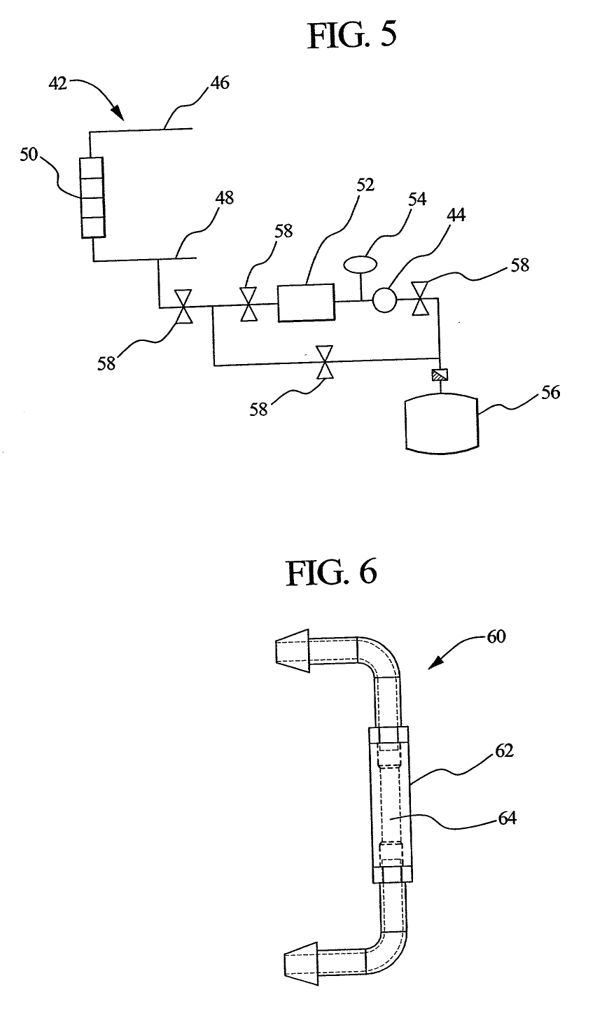

Referring to FIG. 5, an embodiment of the sensor in a dialysis system 42 is shown. FIG. 5 shows a partial schematic diagram of the dialysis system 42. In this embodiment, the dialysis system 42, for example, a peritoneal dialysis system, has a total ammonia and ammonium sensor 44. Fluid paths 46, 48 of the dialysis system 42 are connected to the rest of the dialysis system which is used to dialyze the patient with dialysate.

In the dialysis system 42, the dialysate in the fluid path 46 passes through a sorbant cartridge 50. Urea is removed from the dialysate by converting the urea to ammonia and ammonium (NH₃/NH₄⁺) by a urea catalysis. For example, urease can be used for the urea conversion. The ammonia and ammonium are then removed from the dialysate. For example, ammonia and ammonium absorption agents in the sorbant cartridge 50 remove the ammonia and ammonium from the dialysate. For example, the cationic exchanger zirconium phosphate can be used to remove the ammonium from the dialysate.

In the dialysis system 42, the pH of the solution entering the cartridge 50 can be adjusted to an ammonia/ammonium equilibrium point which reduces the ammonia and increases the ammonium. The sorbants in the cartridge 50 then remove the ammonium from the solution.

Although the ammonia and ammonium are normally removed by the sorbant cartridge 50, the sensor 44 of the dialysis system 42 monitors the fluid for ammonia/ammonium concentrations to confirm that the ammonia and ammonium are being removed and remain below threshold levels. Monitoring the ammonia/ammonium concentration in the dialysate can provide an indication of the effectiveness of ammonia/ammonium removal, exhaustion of the removal capacity of the zirconium phosphate, and a failure of the removal process, for example.

Still referring to FIG. 5, in a further embodiment, dialysate fluid exiting the cartridge 50 in the fluid path 48 can flow through a pH adjuster or conditioner 52 (such as magnesium oxide MgO) to the total ammonia and ammonium sensor 44. The pH adjuster 52 adjusts the pH of the fluid to a known value, for example, about 10 pH. Alternatively or in addition to the pH adjuster 52, a pH sensor could be provided to determine the pH of the fluid. A temperature sensor 54 can be provided to measure the temperature of the fluid at the total ammonia and ammonium sensor 44. In this embodiment, the output of the color changing hydrophobic membrane/photo-detector, the output of the fluid temperature sensor, and the known or sensed fluid pH are used by the controller to determine the total ammonia and ammonium concentration in the dialysate fluid.

As shown in FIG. 5, the fluid exiting the total ammonia and ammonium sensor 44 can be supplied to a drain bag 56. Alternatively, the fluid exiting the sensor 44 can be provided to any other type of drain. In a further alternative and merely by way of example, the fluid exiting the sensor 44 could be provided to any other portion of the dialysis system 42, such as the fluid path 48. Various valves 58 can be provided to control the direction of the fluid flow.

Another alternative embodiment of the total ammonia and ammonium sensor 44 utilizes a pH sensor instead of the pH adjuster 52. The pH sensor measures the fluid pH which is used by the control circuit 34 (FIG. 3) to determine the total ammonia and ammonium concentration of the fluid. The pH sensor can be located either upstream or downstream of the sensor 44, or at the same location as the sensor 44. This embodiment having the pH sensor may be preferred if, for example, it is desired that the fluid exiting the ammonia and ammonium sensor 44 is not pH adjusted. For example, if the fluid exiting the sensor 44 is to be returned to the patient, then it may be desired to maintain the fluid pH a physiologic level.

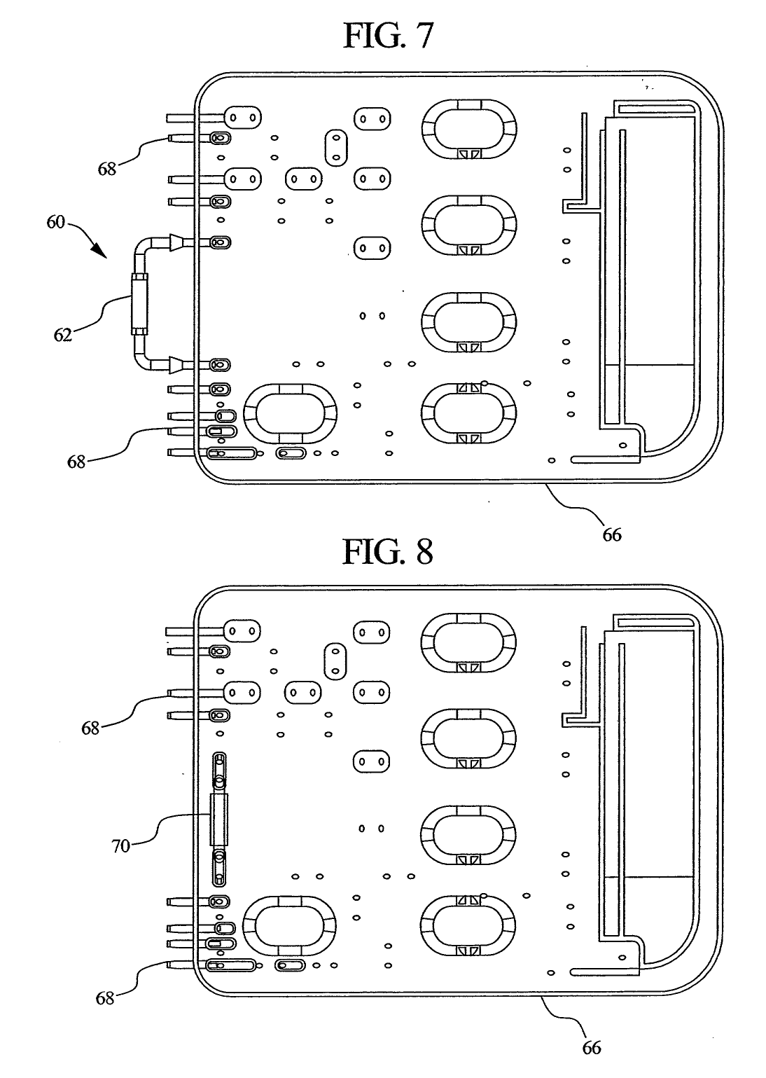

Referring to FIGS. 6 and 7, an example of the pH adjuster 52 (FIG. 5) is shown as pH adjuster 60. The pH adjuster 60 of FIGS. 6 and 7 is a hollow tube 62 having MgO 64 inside of the tube 62. The tube 62 is connected to a fluid pumping cassette 66 which are part of a disposable set for the dialysis system 42. Fluid to be tested for ammonia and ammonium flows from the cassette 66 through the MgO 64 in the tube 62 and back to the cassette 66 to be provided to the hydrophobic membrane. The fluid exiting the MgO 64 is at a known pH, such as about 9.8 pH, for example. At high pH levels, such as approaching a pH of 10, ammonia is maximized and ammonium is minimized in accordance with ammonia/ammonium equilibrium relationships. The maximized ammonia can enhance the color change of the sensor membrane. Of course, other pH levels can be used for the pH adjuster.

The pH adjuster can have any structure and be positioned at any appropriate location rather than as the tube 62 outside of the cassette 66 shown in FIG. 7. For example, referring to FIG. 8, a pH adjuster 70 can be provided within the cassette 66. In another embodiment, the pH adjuster can even be located within the ammonia sensitive membrane itself.

The disposable cassette 66 of FIGS. 7 and 8 is a single use disposable unit for a dialysis therapy treatment. The disposable cassette 66 has a sealed fluid flow path through the cassette 66 with various fluid inlets and outlets 68. The sensor membrane is positioned within the cassette fluid flow path. Other portions of the ammonia sensor, such as the LED's, the optical reader, and the control circuit, are located outside of the disposable cassette 66, for example in an automated dialysis therapy instrument. In this manner, the instrument portion of the sensor can be reused for multiple dialysis treatments, each treatment using a new disposable unit having a new sensor membrane.

One alternative to sensing ammonia with the ammonia sensitive membrane is to sense the ammonium concentration in the fluid. An ionic sensor can be used to sense ammonium in fluid, for example. Of course, the present inventions also pertains to sensing fluid components other than ammonia and ammonium by utilizing the appropriate component indicator and reader.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A sensor for sensing ammonia in a fluid, comprising:
a fluid flow path having an optical window;
a membrane positioned within the fluid flow path, the membrane exhibiting a color indicative of the concentration of the ammonia in the fluid; and
an optical reader positioned outside of the fluid flow path that can identify the color of the membrane through the optical window.

2. The sensor of claim 1, wherein the membrane is a hydrophobic membrane.

3. The sensor of claim 1, further comprising a fluid pH conditioner in the fluid flow path.

4. The sensor of claim 1, further comprising a fluid parameter sensor having an output signal provided to a processor, the processor utilizing the output signal of the fluid parameter sensor to determine the ammonia concentration.

5. The sensor of claim 4, wherein the parameter sensor senses a parameter selected from the group consisting of a temperature, pH, and combinations thereof.

6. The sensor of claim 1, wherein the fluid flow path is a portion of a dialysis system flow path.

7. The sensor of claim 1, wherein the optical window comprises a flexible sheeting.

8. The sensor of claim 1, wherein the optical reader further comprises:
an infrared emitter connected to a processor;
a first color emitter connected to the processor;
a second color emitter connected to the processor; and
a photo-detector connected to the processor.

9. The sensor of claim 1, further comprising a processor which determines a total ammonia and ammonium concentration of the fluid.

10. A sensor for a dialysis system, comprising:
a fluid container for containing a dialysate fluid;
a membrane positioned inside of the fluid container and having a variable optical property; and
an optical reader positioned outside of the fluid container in a sensing relationship with the membrane.

11. The sensor of claim 10, wherein the fluid container is a disposable unit for use in a single dialysis therapy treatment.

12. The sensor of claim 10, wherein the membrane is a hydrophobic membrane.

13. The sensor of claim 10, wherein the membrane is a colorimetric ammonia sensitive membrane.

14. The sensor of claim 10, wherein the optical reader is a colorimetric reader.

15. The sensor of claim 10, further comprising a fluid pH adjustor upstream of the membrane.

16. The sensor of claim 10, further comprising a fluid temperature sensor at the fluid container.

17. The sensor of claim 10, further comprising a processor connected to the optical reader, the processor having an output indicative of a fluid parameter sensed by the sensor.

18. The sensor of claim 17, wherein the output of the processor is indicative of one of ammonia in the fluid flow path, ammonium in the fluid flow path, total ammonia and ammonium in the fluid flow path, and combinations thereof.

19. A sensor for sensing concentrations of a component of a fluid of a dialysis system in which at least a portion of a fluid flow path of the dialysis system is closed to surrounding environment, the sensor comprising:
an optical indicator positioned within the closed fluid flow path and in direct contact with the fluid when the optical indicator is in use, the optical indicator having a variable optical characteristic of the concentration of the component when the optical indicator is in direct contact with the fluid; and
an optical reader located outside of the closed fluid flow path and so positioned and arranged to detect the optical characteristic of the optical indicator, the optical reader generating an output signal indicative of the optical characteristic of the optical indicator.

20. The sensor of claim 19, further comprising a fluid conditioner at one of either upstream of the optical indicator and within the optical indicator.

21. The sensor of claim 20, wherein the fluid conditioner is a pH adjustor.

22. The sensor of claim 19, wherein the variable optical characteristic comprises variable colors.

23. The sensor of claim 19, wherein the optical sensor comprises an ammonia sensing membrane.

24. The sensor of claim 19, wherein the optical reader is a colorimetric sensor.

25. The sensor of claim 19, further comprising a processor which receives the output signal of the optical reader and determines an ammonia concentration based at least in part on the output signal of the optical reader.

26. The sensor of claim 25, further comprising a fluid parameter sensor having an output signal provided to the processor, the processor utilizing the output signal of the fluid parameter sensor in the determination of the ammonia concentration.

27. The sensor of claim 26, wherein the parameter sensor is selected from the group consisting of a temperature sensor, a pH sensor, and combinations thereof.

28. An ammonia sensor for a dialysis system, comprising:
a disposable unit having a fluid flow path;
a ammonia sensitive membrane inside of the fluid flow path; and
a membrane reader positioned outside of the fluid flow path in sensing relationship with the membrane.

29. The ammonia sensor of claim 28, wherein the membrane is a calorimetric ammonia sensitive hydrophobic membrane.

30. The ammonia sensor of claim 28, wherein the membrane reader is a colorimetric reader.

31. A method of sensing ammonia in a dialysis system, comprising the steps of:
providing an ammonia sensitive device inside of a fluid flow path having a fluid inlet and a fluid outlet;
flowing dialysate through the fluid flow path;
allowing the ammonia sensitive device to contact dialysate located in the fluid flow path;
causing a color of a portion of the ammonia sensitive device to change in response to a concentration of ammonia in the dialysate; and
identifying the color of the ammonia sensitive device from outside of the fluid flow path.

32. The method of claim 31, further comprising the step of determining a total ammonia and ammonium concentration of the dialysate is based at least in part on the color of the ammonia sensitive device.

33. The method of claim 31, wherein the step of providing an ammonia sensitive device further comprises the step of providing a hydrophobic ammonia sensitive membrane inside of the fluid flow path.

34. The method of claim 31, further comprising the step of adjusting a pH of the dialysate upstream of the ammonia sensitive device.

35. The method of claim 32, further comprising the step of adjusting a pH of the dialysate upstream of the ammonia sensitive device, and wherein the determining step further comprises the step of determining the total ammonia and ammonium concentration of the dialysate based at least in part on the adjusted pH.

36. The method of claim 35, further comprising the step of measuring a temperature of the dialysate, and wherein the determining step further comprises the step of determining the total ammonia and ammonium concentration of the dialysate based at least in part on the measured temperature.

37. The method of claim 32, further comprising the step of measuring a pH of the dialysate, and wherein the determining step further comprises the step of determining the total ammonia and ammonium concentration of the dialysate based at least in part on the measured pH.

38. The method of claim 37, further comprising the step of measuring a temperature of the dialysate, and wherein the determining step further comprises the step of determining the total ammonia and ammonium concentration of the dialysate based at least in part on the measured temperature.

39. A method of performing dialysis, comprising the steps of:
removing waste from a patient using dialysate fluid;
positioning in the dialysate fluid a membrane that changes a parameter in relation to the level of a component in the dialysate fluid; and
identifying the change in the parameter of the membrane.

40. The method of performing dialysis of claim 39, wherein the positioning step further comprises contacting an ammonia sensitive membrane with the dialysate fluid.

41. The method of claim 39, further comprising changing an optical parameter of the membrane.

42. The method of claim 39, wherein the identifying step further comprises sensing a color of the membrane.

43. The method of claim 39, further comprising the step of treating the dialysate fluid prior to the step contacting the membrane with the dialysate fluid.

44. The method of claim 43, wherein the treating step further comprises adjusting a pH of the dialysate fluid.

45. The method of claim 39, wherein the step of removing waste further comprises performing peritoneal dialysis.

46. The method of claim 39, wherein the step of removing waste further comprises performing hemodialysis.

47. The method of claim 39, wherein the component is ammonia.

48. The method of claim 39, wherein the component is ammonium.

49. A method of performing dialysis, comprising the steps of:
removing waste from a patient using dialysate fluid and thereby forming spent dialysate;
positioning in the spent dialysate an ammonia sensitive member which has a characteristic that changes in relation to the level of ammonia in the spent dialysate; and
identifying a change in the characteristic of the ammonia sensitive member.

50. The method of performing dialysis of claim 49, wherein the ammonia sensitive member is a membrane.

51. The method of claim 49, wherein the characteristic of the member is color.

52. The method of claim 49, wherein the step of removing waste further comprises performing peritoneal dialysis.

53. The method of claim 49, wherein the step of removing waste further comprises performing hemodialysis.
